(19) United States
(12) Patent Application Publication
(10) Pub. No.: US 2020/0355721 A1
(43) Pub. Date: Nov. 12, 2020

(54) METHOD AND APPARATUS FOR DETECTING WALKING FACTOR WITH PORTION ACCELERATION SENSOR

(71) Applicant: Beflex Inc., Daejeon (KR)

(72) Inventor: Chang Keun JUNG, Daejeon (KR)

(21) Appl. No.: 16/838,992

(22) Filed: Apr. 2, 2020

(30) Foreign Application Priority Data
May 8, 2019 (KR) 10-2019-0053477

(51) Int. Cl. G01P 13/00; G01P 15/00; G01S 19/01; A61B 5/11; A61B 5/00

(52) U.S. Cl. CPC G01P 13/00; G01P 15/00; G01S 19/01; A61B 5/6823; A61B 5/112; A61B 5/6803; A61B 5/7278; A61B 5/1114

(57) ABSTRACT

Provided is a method of detecting a gait parameter through a head part acceleration sensor. The method of detecting a gait parameter through a head part acceleration sensor includes: a gait acceleration collecting step; a gait point-in-time detecting step; and a gait parameter detecting step, wherein the start point and the end point of the double-limb support are set within a window predetermined on the basis of peaks of the gait cycle, the start point of the double-limb support is the same as an end point of a single-limb support, and the end point of the double-limb support is the same as a start point of a single-limb support.

### CROSS-REFERENCE TO RELATED APPLICATIONS

This application claims priority under 35 U.S.C. § 119 to Korean Patent Application No. 10-2019-0053477, filed on May 8, 2019, in the Korean Intellectual Property Office, the disclosure of which is incorporated herein by reference in its entirety.

### TECHNICAL FIELD

The following disclosure relates to a method and an apparatus of detecting a gait parameter through an acceleration sensor.

### BACKGROUND

Recently, development for a system that analyzes physical activities of a person in daily life using various sensors has been actively conducted. A gait among the physical activities of the person has been known as a natural movement that hardly requires cerebration, but has been found to be actually related to high-level cognitive functions such as concentration and executive ability.

Therefore, a gait analysis of the person has been utilized as an important measure for evaluating whether or not a subject may maintain normal daily life.

### SUMMARY

The present applicant has found that it is possible to analyze a gait posture of a pedestrian by attaching only an acceleration sensor to a head part of the pedestrian without using the force plates through an experiment. In addition, the present applicant has found that in a case of analyzing the gait posture by attaching the acceleration sensor to the head part, accuracy of the analysis is improved more effectively than a case of attaching acceleration sensors to limb parts.

Aspects of the invention provide a method and an apparatus of detecting a gait parameter through a head part acceleration sensor capable of improving analysis accuracy for a gait posture by collecting a gait acceleration signal using an acceleration sensor attached to a head part of a pedestrian to detect a gait point-in-time and a gait parameter. More specifically, an embodiment of the present invention is directed to providing a method of detecting a gait parameter through a head part acceleration sensor capable of improving a working velocity by collecting a gait acceleration signal of a pedestrian and analyzing a gait posture of the pedestrian using a head part acceleration sensor attached to a head part of the pedestrian.

Another embodiment of the present invention is directed to providing a method of detecting a gait parameter through a head part acceleration sensor capable of increasing convenience of a work by distinguishing a left foot and a right foot of a pedestrian from each other on the basis of a gait acceleration collected from the head part acceleration sensor.

In one general aspect, a method of detecting a gait parameter through a head part acceleration sensor includes: a gait acceleration collecting step of collecting an acceleration in a vertical direction of a pedestrian measured by an acceleration sensor disposed at a head part of the pedestrian; a gait point-in-time detecting step of setting a gait point-in-time by extracting a peak value in the acceleration in the vertical direction within a gait cycle including a standing phase and a swing phase and detecting a start point and an end point of a double-limb support on the basis of the peak value; and a gait parameter detecting step of detecting the double-limb support on the basis of the start point and the end point in order to analyze a gait posture of the pedestrian, wherein the start point and the end point of the double-limb support are set within a window predetermined on the basis of peaks of the gait cycle, the start point of the double-limb support is the same as an end point of a single-limb support, and the end point of the double-limb support is the same as a start point of a single-limb support.

In the gait point-in-time detecting step, a gait jerk signal at a point-in-time in which an impact is applied to a ground when a limb moves from the single-limb support to the double-limb support may be further detected by differentiating the acceleration in the vertical direction, and a peak value of the gait jerk signal located before the peak value of the acceleration in the vertical direction may be set as the start point of the double-limb support and a peak value of the gait jerk signal located after the peak value of the acceleration in the vertical direction may be set as the end point of the double-limb support.

In the gait parameter detecting step, a cadence and a step width may be detected on the basis of the peak value of the acceleration in the vertical direction, and a gait velocity, a stride length, and a step length of the pedestrian may be further detected in a case of using a global positioning system (GPS).

The stride length may be calculated by the following Equation 1:

Stride Length=Gait Velocity/Cadence. (Equation 1)

The gait acceleration collecting step may include a step of collecting an acceleration in a horizontal direction of the pedestrian, and a left foot and a right foot of the pedestrian may be distinguished from each other according to signs of the acceleration in the horizontal direction.

In the window, after a highest peak of the peaks of the gait cycle is set as a reference peak, a window size may be set to be smaller than a range between peaks disposed on both sides of the reference peak.

In another general aspect, an apparatus of detecting a gait parameter through a head part acceleration sensor includes: a gait measuring module measuring an acceleration in a vertical direction by a gait acceleration sensor disposed in the vicinity of a head of a pedestrian and transferring a measured value; and a gait analyzing module including an acceleration collecting module collecting the measured value received from the gait measuring module, a gait point-in-time module extracting a peak value in the acceleration in the vertical direction on the basis of the measured value and detecting a start point and an end point of a double-limb support, and a gait parameter detecting module detecting the double-limb support on the basis of the start point and the end point, wherein the start point and the end point of the double-limb support are set within a window predetermined on the basis of peaks of a gait cycle, the start point of the double-limb support is the same as an end point of a single-limb support, and the end point of the double-limb support is the same as a start point of a single-limb support.

The gait point-in-time module may further detect a gait jerk signal at a point-in-time in which an impact is applied to a ground when a limb moves from the single-limb support to the double-limb support by differentiating the acceleration in the vertical direction, and a peak value of the gait jerk signal located before the peak value of the acceleration in the vertical direction may be set as the start point of the double-limb support and a peak value of the gait jerk signal located after the peak value of the acceleration in the vertical direction may be set as the end point of the double-limb support.

The apparatus of detecting a gait parameter through a head part acceleration sensor may further include a notifying module converting the gait parameter detected by the gait analyzing module into a form recognized by a user, including a sound or an image, and outputting gait posture analysis information so that the pedestrian corrects a gait posture.

Detailed contents of other exemplary embodiments are described in a detailed description and are illustrated in the accompanying drawings.

### BRIEF DESCRIPTION OF THE DRAWINGS

FIGS. 1A to 1B are schematic views illustrating an apparatus of detecting a gait parameter through a head part acceleration sensor according to an exemplary embodiment of the present invention.

FIG. 2 is a configuration diagram of the apparatus of detecting a gait parameter according to an exemplary embodiment of the present invention.

FIG. 3 is a schematic flowchart of a method of detecting a gait parameter through a head part acceleration sensor according to an exemplary embodiment of the present invention.

FIG. 4 is a detailed flowchart of the method of detecting a gait parameter according to an exemplary embodiment of the present invention.

FIG. 5 is an illustrative view for describing a process of detecting a gait point-in-time by collecting a gait acceleration signal according to an exemplary embodiment of the present invention.

FIG. 6 is an illustrative view for describing a process of detecting the highest point, the lowest point, and a jerk of the collected gait acceleration signal according to an exemplary embodiment of the present invention.

FIGS. 7 and 8 are illustrative views for describing a process of setting a window on the basis of the highest point of the gait acceleration signal according to an exemplary embodiment of the present invention.

FIG. 9 is an illustrative view for describing a process of detecting a start point and an end point of a double-limb support at a jerk in the window according to an exemplary embodiment of the present invention.

FIG. 10 is an illustrative view for describing a process of distinguishing a left foot and a right foot from each other depending on the gait acceleration signal according to an exemplary embodiment of the present invention.

FIG. 11 is a view for describing a double-limb support and a single-limb support according to an exemplary embodiment of the present invention.

FIG. 12 is a view for describing a process of analyzing a gait posture of a user on the basis of peak values in region A of FIG. 11.

FIG. 13 is an illustrative view illustrating the gait posture of the user through FIG. 12 in detail.

FIGS. 14A to 14D are graphs illustrating ground reaction forces for each time according to another exemplary embodiment of the present invention.

### DETAILED DESCRIPTION OF EMBODIMENTS

Hereinafter, various exemplary embodiments of the present invention will be described in detail with reference to the accompanying drawings.

The following description exemplifies only a principle of the present invention. Therefore, those skilled in the art may invent various apparatuses implementing the principle of the present invention and included in the spirit and scope of the present invention although not clearly described or illustrated in the present specification. In addition, it is to be understood that all conditional terms and exemplary embodiments mentioned in the present specification are basically intended only to allow those skilled in the art to understand a concept of the present invention, and the present invention is not limited to exemplary embodiments and states particularly mentioned as such.

In addition, in the following description, ordinal expressions such as first and second are intended to describe objects that are equivalent to and are independent of each other, and should be understood to have no meaning of main/sub or master/slave in the order.

The aspects, features, and advantages described above will become more obvious from the following detailed description provided in relation to the accompanying drawings. Therefore, those skilled in the art to which the present invention pertains may easily practice the technical spirit of the present invention.

Features of each of several exemplary embodiments of the present invention may be partially or entirely coupled to and combined with each other, may technically variously interwork and be driven with each other as sufficiently understood by those skilled in the art, and the respective exemplary embodiments may be implemented independently of each other or may be implemented together in an association relationship.

An apparatus of measuring a gait motion according to an implementation directly measures ground reaction forces by attaching sensors to limb parts of a human body or has collected ground reaction forces separated from each other from two different force plates. Since the apparatus of measuring a gait motion separately collects the ground reaction forces for the left foot and the right foot from the two force plates as described above, an expensive equipment should be purchased in order to analyze a gait posture of a pedestrian. Furthermore, in a case of collecting the ground reaction forces using the force plates, portability may be poor.

There may be an attempt to measure running using an acceleration sensor. However, since a person moves at a high velocity at the time of the running, only acceleration signals for the left foot and the right foot are mainly measured, and a double-limb support hardly appear. Therefore, it would be very difficult to analyze a single-limb support or a separate movement of the left foot or the right foot using an acceleration sensor.

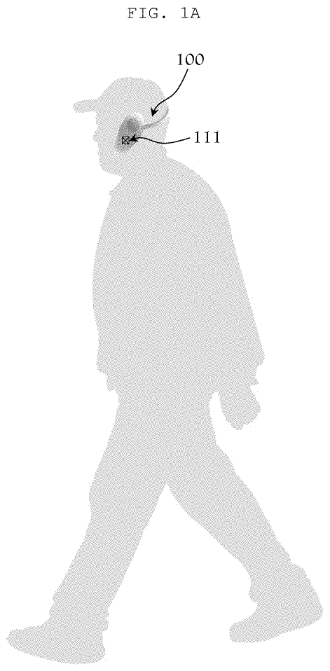

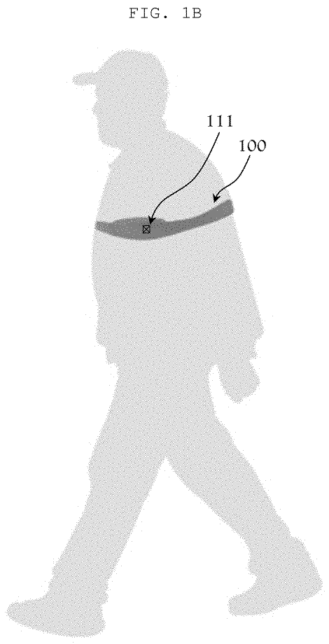

In embodiments of the invention, a configuration of an apparatus 100 of detecting a gait parameter according to an exemplary embodiment of the present invention and a method of detecting a gait parameter using the same will be described with reference to FIGS. 1A to 4.

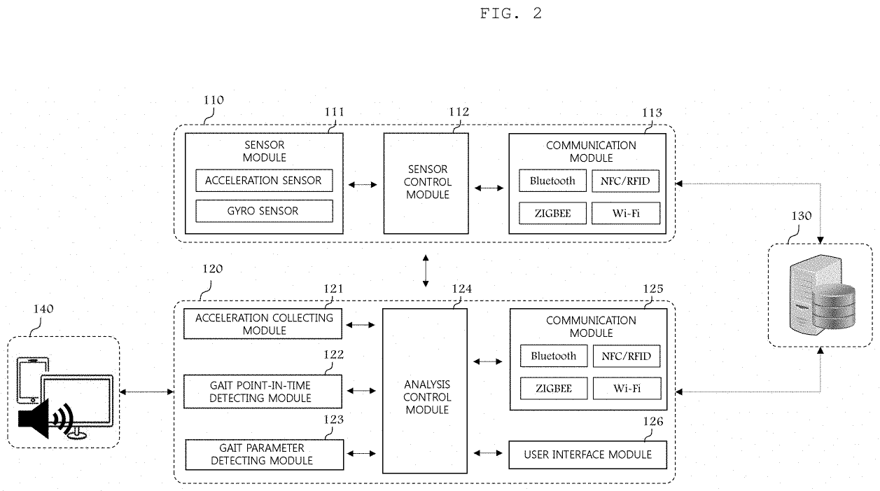

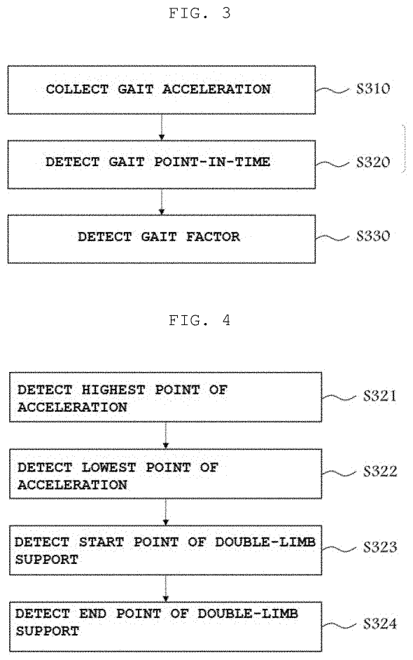

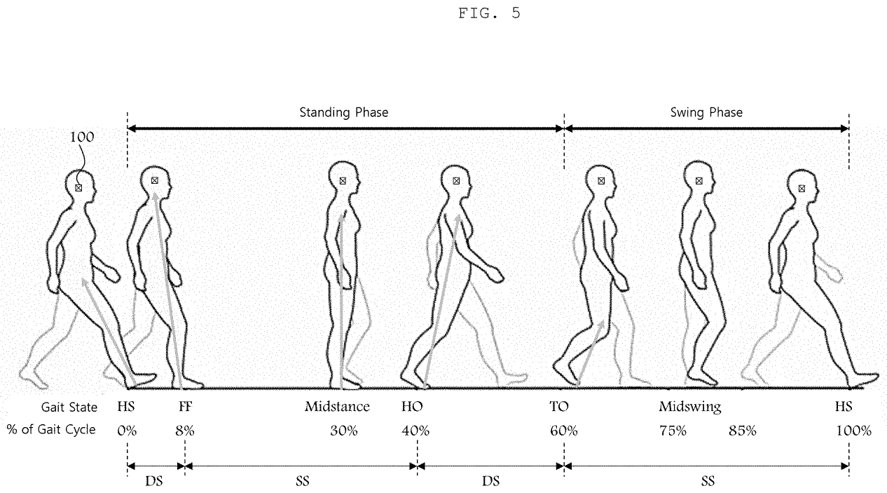

FIGS. 1A to 1B are schematic views illustrating an apparatus of detecting a gait parameter through a head part acceleration sensor according to an exemplary embodiment of the present invention. FIG. 2 is a configuration diagram of the apparatus of detecting a gait parameter according to an exemplary embodiment of the present invention. FIG. 3 is a schematic flowchart of a method of detecting a gait parameter through a head part acceleration sensor according to an exemplary embodiment of the present invention. FIG. 4 is a detailed flowchart of the method of detecting a gait parameter according to an exemplary embodiment of the present invention.

Referring to FIGS. 1A and 1B, an apparatus 100 of detecting a gait parameter is an apparatus that may accurately analyze a gait motion by collecting a gait acceleration of a pedestrian when the pedestrian walks, and is an acceleration sensor attached to a headset, an earphone, a hat, and the like, which may be worn on a head of the pedestrian. In embodiments of the present invention, the apparatus 100 of detecting a gait parameter is basically attached to the headset to measure the gait parameter, as illustrated in FIG. 1A. However, the apparatus 100 of detecting a gait parameter is not limited thereto, and may be worn on the upper body in the vicinity of the waist or the chest of a user (see FIG. 1B).

The apparatus 100 of detecting a gait parameter includes a gait measuring module 110, a gait analyzing module 120, a database 130, and a notifying module 140, as illustrated in FIG. 2.

The gait measuring module 110 measures a gait acceleration signal of the pedestrian using an acceleration sensor attached to a head part of the pedestrian, and is attached to the headset. However, the gait measuring module 110 is not limited thereto, and may be detached from and attached to the chest part of the pedestrian (see FIG. 1B) using a band or be attached to the vicinity of the head or the vicinity of the upper body of the pedestrian. For example, the gait measuring module 110 may be attached to the vicinity of the upper arm, the ear, the head, and the shoulder of the pedestrian using a band or various apparatuses. However, it is preferable that the gait measuring module 110 is attached to a part close to the head part except for the lower body, because accuracy of measurement is decreased in a case where the gait measuring module 110 is attached to and detached from a body part below the upper body. In addition, in the present invention, the gait measuring module 110 may be accommodated in a housing separate from a housing in which the gait analyzing module 120 is accommodated or may be accommodated in the same housing as a housing in which the gait analyzing module 120 is accommodated.

Referring to FIG. 2, the gait measuring module 110 may include a sensor module 111, a sensor control module 112 and a communication module 113.

The sensor module 111 may measure sensor values required for a gait analysis of the gait analyzing module 120 and transmit the measured sensor values to the gait analyzing module 120. It has been described in the present invention that the sensor module 111 senses only the sensor values required for the gait analysis, but the sensor module 111 may detect a gait point-in-time and a gait parameter by collecting a sensed acceleration and transmit the detected gait point-in-time and gait parameter to the gait analyzing module 120. In addition, the sensor module 111 may include an acceleration sensor and a gyro sensor, or may perform measurement using a global positioning system (GPS) sensor. A detailed measuring method related to the sensor module will be described later. In addition, the sensor control module 112 may control a general operation of the gait measuring module 110.

The communication module 113 may transmit the sensor values sensed by the sensor module 111 to the gait analyzing module 120. The communication module 113 may include, for example, a Bluetooth module, a near field communication (NFC) module, a radio frequency identification (RFID) module, a Zigbee module, a wireless fidelity (Wi-Fi) module, and the like.

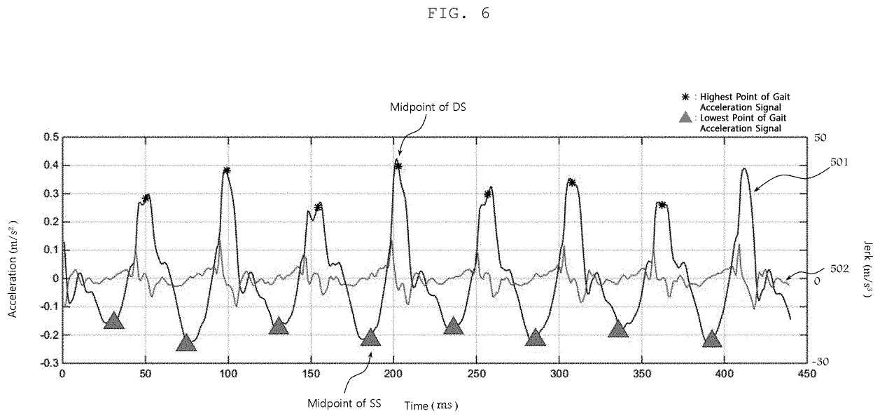

The gait analyzing module 120 includes an acceleration collecting module 121 receiving a gait acceleration signal from the sensor module 111 of the gait measuring module 110 and collecting a gait acceleration, a gait point-in-time detecting module 122 detecting a gait point-in-time by detecting the lowest point and the highest point of the gait acceleration from the collected gait acceleration, and a gait parameter detecting module 123 deriving a gait parameter on the basis of the detected gait point-in-time. In addition, a communication module 125 may be provided in order to transmit a signal to the gait measuring module 110 and the notifying module 140. Detailed operations of the gait point-in-time detecting module 122 and the gait parameter detecting module 123 will be described later with reference to FIGS. 6 to 13.

Meanwhile, in a case where the acceleration collecting module 121 is formed integrally with the gait measuring module 110, the communication module 113 of the gait measuring module 110 may be directly connected to the gait analyzing module 120 to transmit the collected gait acceleration signal to the gait analyzing module 120. In addition, such information on the gait analysis may be output through the notifying module 140. Such information may be output by, for example, a speaker, a mobile phone, a computer, a wireless earphone, or the like.

The notifying module 140 converts gait posture analysis information generated by the gait analyzing module 120 into information that may be recognized by a user, such as a sound or an image, and outputs the converted information. For example, in a case where a stride correction is necessary, the notifying module 140 may output a voice such as “reduce a stride” or a warning sound such as “beep” through a speaker provided in the apparatus 100 of detecting a gait parameter or a mobile phone, a computer, and a wireless earphone connected to the apparatus 100 of detecting a gait parameter. Here, in a case where a stride to be corrected by the user is long, the warning sound is quickly output, and in a case where a stride to be corrected by the user is short, the warning sound is slowly output, such that the user may recognize how much the stride needs to be corrected with only the warning sound and correct the stride. Particularly, it has been mentioned that the wireless earphone or the speaker may be included in the notifying module 140, but the acceleration sensor is basically attached to the earphone worn on the head part of the user in the present invention, and the gait posture analysis information may thus be output directly to ears of the user through the apparatus 100 of detecting a gait parameter. In addition, the notifying module 140 may be realized in various forms, such as being connected to a smart phone, a computer, a dedicated display, or the like, to output accurate correction information as an image.

In addition, the apparatus 100 of detecting a gait parameter may transmit and store the gait posture analysis information derived by the gait analyzing module 120 to and in the database 130. Here, the gait posture analysis information may be cumulatively stored, such that the gait posture of the pedestrian may be confirmed according to a change in time. Therefore, when a large amount of gait posture analysis information is cumulatively stored, such data may be variously utilized. For example, such data may be utilized as big data to be used for various statistics and analyses. An analysis control module 124 of the apparatus 100 of detecting a gait parameter may control a general operation of the gait analyzing module 120 described above.

The apparatus 100 of detecting a gait parameter is attached to the head part of the pedestrian to collect the gait acceleration (S310). Then, the apparatus 100 of detecting a gait parameter detects the gait point-in-time (S320).

The gait point-in-time refers to the highest point or the lowest point of the acceleration or a start point and an end point of a single-limb support or a double-limb support, detected from the gait acceleration signal collected in order to detect the gait parameter. Here, the gait acceleration refers to an acceleration that changes during a period in which the pedestrian walks, and a gait cycle of the pedestrian may be determined on the basis of the gait acceleration. For example, as illustrated in FIG. 4, the gait acceleration of the pedestrian is calculated by measuring each of accelerations depending on the gait cycle.

Referring to FIG. 4, the gait cycle may be divided into a standing phase and a swing phase. The standing phase is a phase in which a body weight is loaded while a foot comes into contact with the ground, and may also be defined as a posture step. The swing phase is a phase in which a limb is away from the ground and moves forward, and may also be defined as a swing step. In this case, the body weight is loaded on the other limb. As illustrated in FIG. 4, the standing phase occupies about 60% of the gait cycle and the swing phase occupies the remaining 40% of the gait cycle.

A section where both feet come into contact with the ground at the same time during the gait cycle is called a double-limb support (DS), and appears twice during one gait cycle. In addition, a section in which only one foot comes into contact with the ground during the gait cycle is called a single-limb support (SS), and appears twice during one gait cycle. In this case, as a gait velocity of the pedestrian become fast, the single-limb support (SS) may be increased and the double-limb support (DS) may be decreased.

Specifically, the standing phase includes gait states of five steps such as a heel strike (HS), a foot flat (FF), a midstand, a heel off (HO), and a toe off (TO). In addition, the swing phase includes gait states of three steps such as a toe off (TO), midswing, and a heel strike (HS). In the present specification, the gait state has been described on the basis of the right foot, and a description of the left foot has been described as an expression such as an opposite side.

In other words, the standing phase includes an initial contact in which the body weight is loaded while the foot starts to come into contact with the ground, a loading response, a midstance, and a terminal stance in which the body weight is loaded while the feet comes into complete contact with the ground, and a pre-swing, which is a phase immediately before the heel of the foot is lifted from the ground while the body weight is loaded on an opposite limb before entering the swing phase.

The swing phase is divided into an initial swing, a midswing and a terminal swing. Specifically, the initial swing is a phase which starts with a toe off and in which the limb moves forward while acceleration is applied to the limb. The midswing is a phase in which the knee joint is extended and the muscle hardly acts as the limb passes through the opposite limb. The terminal swing may also be defined as a velocity reducing period, and in the terminal swing, and may move on to the initial contact of the standing phase by naturally reducing a velocity of the limb moving forward.

Times required for the respective gait phases described above are constant for each individual, but as a pace becomes faster, the double-limb support may be reduced.

Hereinafter, a process of detecting the gait point-in-time and the gait parameter will be described with reference to FIG. 4 together with FIGS. 5 to 10.

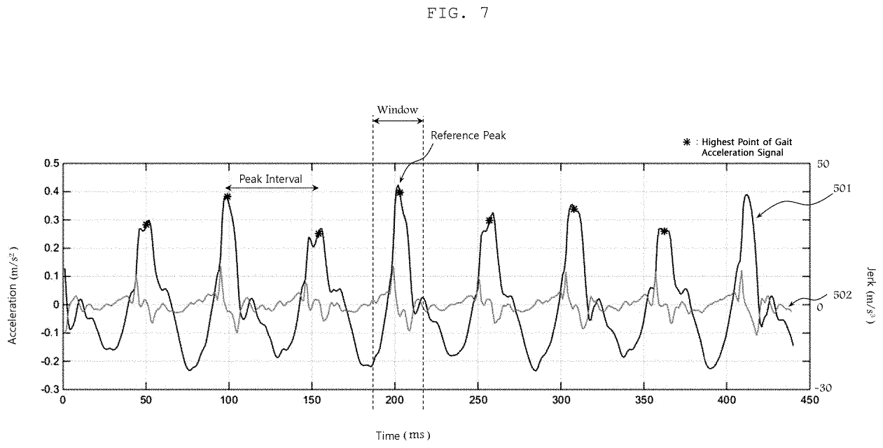

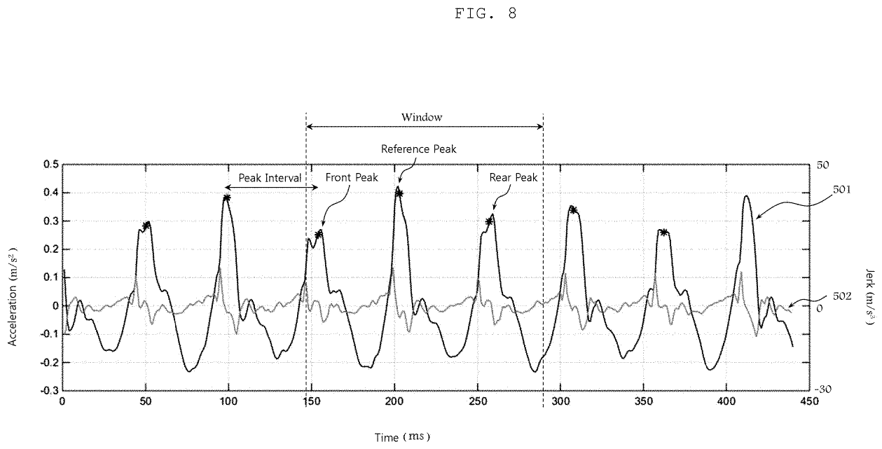

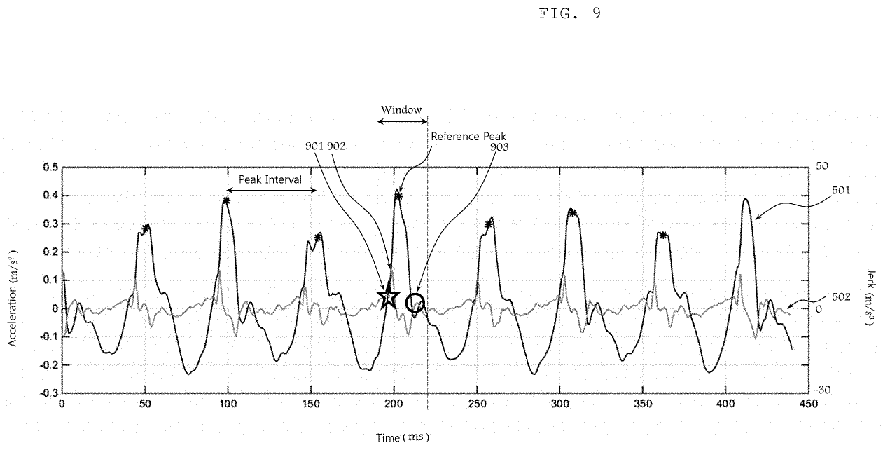

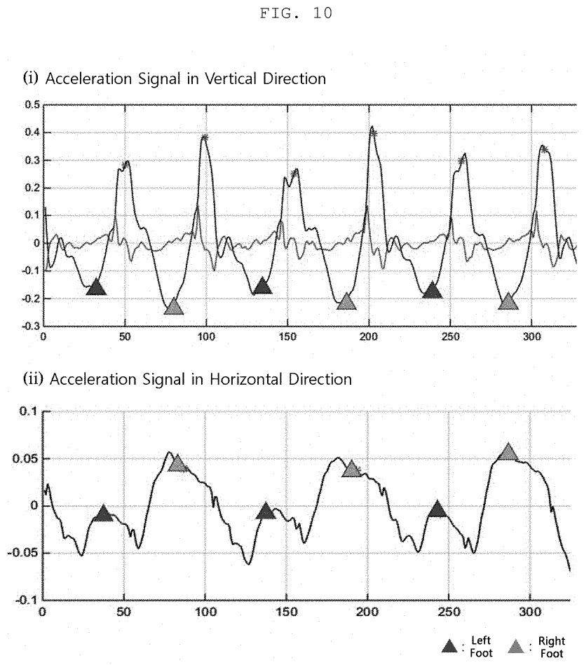

FIG. 5 is an illustrative view for describing a process of detecting a gait point-in-time by collecting a gait acceleration signal according to an exemplary embodiment of the present invention. FIG. 6 is an illustrative view for describing a process of detecting the highest point, the lowest point, and a jerk of the collected gait acceleration signal according to an exemplary embodiment of the present invention. FIGS. 7 and 8 are illustrative views for describing a process of setting a window on the basis of the highest point of the gait acceleration signal according to an exemplary embodiment of the present invention. FIG. 9 is an illustrative view for describing a process of detecting a start point and an end point of a double-limb support at a jerk in the window according to an exemplary embodiment of the present invention. FIG. 10 is an illustrative view for describing a process of distinguishing a left foot and a right foot from each other depending on the gait acceleration signal according to an exemplary embodiment of the present invention.

The gait analyzing module 120 may detect the gait point-in-time of the left foot and the right foot measured by the head part acceleration sensor attached to the head part of the pedestrian on the basis of the collected gait acceleration signal. Specifically, as illustrated in FIGS. 4 and 6, in order to detect the gait point-in-time (S320), the highest point of the acceleration is detected (S321), and the lowest point of the acceleration is detected (S322). Then, the start point of the double-limb support is detected (S323). Then, the end point of the double-limb support is detected (S324).

Specifically, a gait acceleration signal 501 according to a time (ms) is collected using the apparatus 100 of detecting a gait parameter attached to the head part of the pedestrian when the pedestrian walks. In this case, the gait acceleration signal may be an acceleration in a vertical direction, or may be a norm of a signal of one or more axes including the acceleration in the vertical direction. For example, a one-axis (vertical) acceleration, a multi-axis acceleration including a vertical acceleration, or the like may be used. In this case, parameters that may be collected at the time of using the one-axis acceleration are as illustrated in Table 1, and parameters that may be collected at the time of using the multi-axis acceleration are as illustrated in Table 2.

TABLE 1

| Parameter | Unit |
|---|---|
| Cadence | steps/min |
| Stride Duration | ms |
| First peak acceleration | G |
| Trough acceleration | G |
| Second peak acceleration | G |
| First peak force | N |
| (if mass known) | |
| Trough force | N |
| (if mass known) | |
| Second peak force | N |
| (if mass known) | |
| Step count | step |
| Vertical oscillation | m |

TABLE 2

| Parameter | Unit |
|---|---|
| Left Single Support Duration | ms |
| Left Double Support Duration | ms |
| (Left trailing limb) | |
| Left First peak acceleration | G |
| Left Trough acceleration | G |
| Left Second peak acceleration | G |
| Left First peak force | N |
| Left Trough force | N |
| Left Second peak force | N |
| Right Single Support Duration | ms |
| Right Double Support Duration | ms |
| (Right trailing limb) | |
| Right First peak acceleration | G |
| Right Trough acceleration | G |
| Right Second peak acceleration | G |
| Right First peak force | N |
| Right Trough force | N |
| Right Second peak force | N |
| Head angle | deg |
| Step Width | m |

Then, peaks *, which are the highest points in the collected gait acceleration signal 501, are detected, and peaks ▴, which are the lowest points in the collected gait acceleration signal 501, are detected. A peak *, which is the highest value of the peaks *, which are the highest points of the gait acceleration signal 501, indicates a midpoint of the double-limb support (DS), and a peak ▴, which is the lowest value of the peaks ▴, which are the lowest points of the gait acceleration signal 501, indicates a midpoint of the single-limb support (SS).

In addition, by obtaining a gait jerk signal 502, which is a differential value of the gait acceleration signal 501, it is possible to detect a point-in-time in which an impact is applied to the ground when the limb comes into contact with the ground during moving from the single-limb support (SS) to the double-limb support (DS). In addition, the gait jerk signal 502 is necessarily required in order to analyze the gait acceleration signal 501 in more detail. Here, the gait jerk signal 502 may be obtained by detecting peak values in a high pass filter (HPF) extracting a preset high frequency signal, calculating an average value of the detected peak values, and multiplying the average value by a prediction coefficient to calculate a predicted value of a gradient (IVLR) of a vertical ground reaction force.

Here, a unit of the IVLR may be N/s, which is an absolute unit indicating a force per unit time, or may be (body weight (BW) or g)/s, which is a relative unit. Since the sensor according to embodiments of the present invention basically collects acceleration data rather than a force, data is collected in BW/s, which is a relative unit. When g/s is multiplied by a mass (m) of the user, the IVLR may also be expressed in N/s ((N=m*g)/s), which is an absolute unit. In this case, the IVLR may be included in a prediction coefficient (k1 or k2) or may be calculated by prediction coefficient (k1 or k2)×mass (m) of user as a separate coefficient.

Here, the high frequency signal may refer to an acceleration vertical signal having a frequency of 10 Hz or more. An acceleration vertical signal having a frequency of 5 Hz or more is hardly generated by an arbitrary movement of the user and an acceleration vertical signal generated in a situation such as an impact or the like generally exists in a high band. Therefore, in embodiments of the present invention, the high frequency signal having the frequency of 10 Hz or more is intended to be used to predict the gradient (IVLR) of the vertical ground reaction force. However, the present invention is not limited thereto, and high frequency signal bands extracted by filtering may be set to be different from each other and be applied to embodiments of the present invention, if necessary.

Then, a window size is set before detecting the gait parameter. The window size may mean that a certain section is determined temporally among all samples corresponding to the entire gait acceleration signal 501. A method for determining a gait cycle according to embodiments of the present invention ends in a window set for real-time application. Specifically, referring to FIG. 7, a peak is repeated every 50 ms, and it may thus be seen that a peak interval is approximately 50 ms. In this case, a window of n seconds before and after a reference peak *, which is the highest point of the gait acceleration signal 501, is set. In this case, the window may be set to be increased or decreased in proportion to the peak interval (a reciprocal of a cadence and approximately 50 ms) (see FIG. 6). For example, the window size may be set to a value obtained by multiplying the peak interval by 0.6. However, it is preferable that the window size is fixed to a number of 0.1 seconds or more and 0.4 seconds or less, which is the double-limb support.

However, it is to be noted that the window size is too large to be set to include a front peak and a rear peak, as illustrated in FIG. 8.

Therefore, in a case where it is assumed that the window is set before and after the reference peak as illustrated in FIG. 9, a peak 901 (⋆) of the gait jerk signal 502 appearing before the reference peak within a window range is set as the start point of the double-limb support (DS) (or the end point of the single-limb support (SS)). In addition, a peak 903 (∘) of the gait jerk signal 502 appearing after the reference peak is set as the end point of the double-limb support (DS) (or the start point of the single-limb support (SS)). In this case, an inflection point 902 of the gait jerk signal exists between the peak 901 (⋆) of the gait jerk signal 502 and the reference peak. At the infection point 902, a change amount in an acceleration value may be rapidly reduced.

In addition, embodiments of the present invention are based on collecting the acceleration signal in the vertical direction at the time of collecting the gait acceleration, but as illustrated in (i) and (ii) of FIG. 10, an acceleration signal in a horizontal direction may be collected together at the time of collecting the acceleration signal in the vertical direction. Therefore, in embodiments of the present invention, it is possible to distinguish the left foot and the right foot of the pedestrian from each other according to signs of the acceleration signal in the horizontal direction corresponding to peaks ▴ and [symbol] , which are the lowest points of the acceleration signal in the vertical direction. For example, a case where the sign of the acceleration signal in the horizontal direction corresponding to the acceleration signal in the vertical direction is negative (−) may mean the left foot, and a case where the sign of the acceleration signal in the horizontal direction corresponding to the acceleration signal in the vertical direction is positive (+) may mean the right foot. However, the present invention is not limited thereto, and a case where the sign of the acceleration signal in the horizontal direction corresponding to the acceleration signal in the vertical direction is negative (−) may mean the right foot and a case where the sign of the acceleration signal in the horizontal direction corresponding to the acceleration signal in the vertical direction is positive (+) may mean the left foot.

In addition, as illustrated in (ii) of FIG. 10, a step width in the horizontal direction may be calculated using sizes of points corresponding to peaks ▴ and [symbol] of the acceleration signal in the horizontal direction.

Hereinafter, a process of detecting a gait parameter will be described with reference to FIGS. 11 to 13.

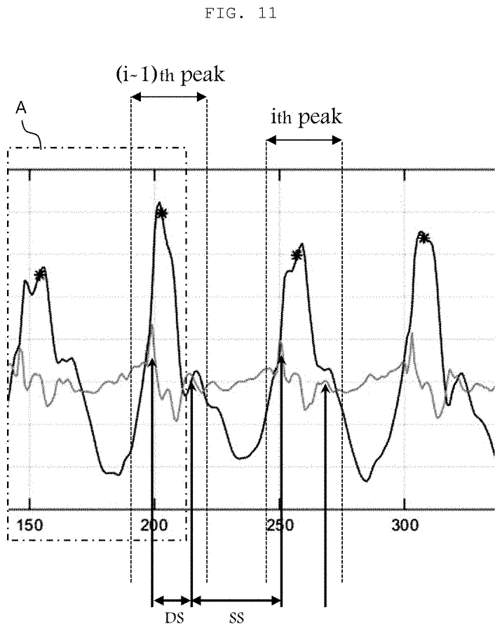

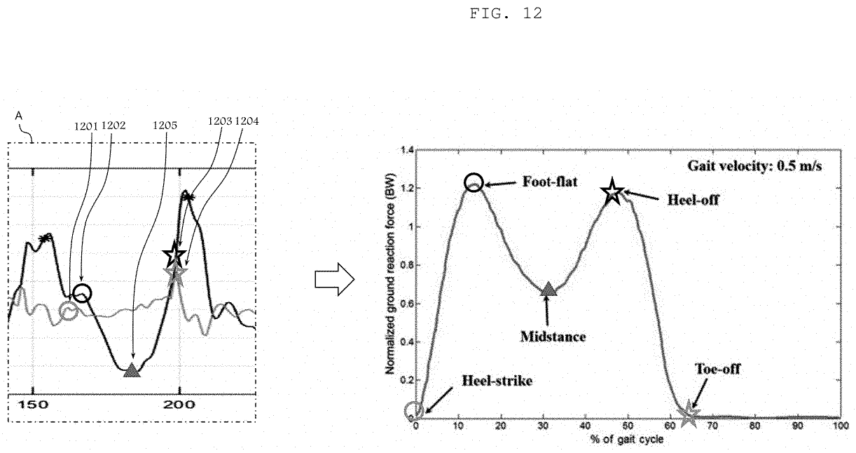

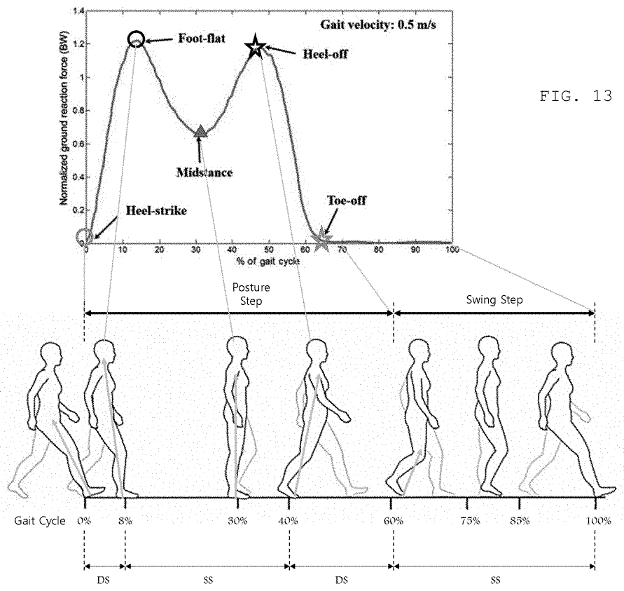

FIG. 11 is a view for describing a double-limb support and a single-limb support according to an exemplary embodiment of the present invention. FIG. 12 is a view for describing a process of analyzing a gait posture of a user on the basis of peak values in region A of FIG. 11. FIG. 13 is an illustrative view illustrating the gait posture of the user through FIG. 12 in detail.

Then, the apparatus 100 of detecting a gait parameter detects the gait parameter (S330). Here, the gait parameter is a component detected in order to analyze the gait posture of the pedestrian on the basis of the detected gait point-in-time, and includes the double-limb support (DS), the single-limb support (SS), a cadence, and a step width. However, the gait parameter is not limited thereto, and may also include force magnitudes of a first peak and a second peak, and a trough force magnitude of the single-limb support within the gait cycle. Therefore, the gait parameter may indicate various components capable of analyzing the gait posture of the pedestrian.

First, referring to FIG. 11, a peak section of the gait jerk signal 502 before and after a peak ((i−1)-th peak) located in the vicinity of a point-in-time of 200 ms is a double-limb support (DS), and a section from a peak of the gait jerk signal after the peak ((i−1)-th peak) to a peak of the gait jerk signal before a peak (i-th peak) located in the vicinity of a point-in-time of 250 ms is a single-limb support (SS). In other words, a time (t DS i) of a double-limb support of the i-th peak is a value obtained by subtracting a time of a start time of the double-limb support from a time of an end point of the double-limb support (DS) (t DS i=t DS end i−t DS start i). In addition, a time of a single-limb support of the i-th peak is a value obtained by subtracting a time of a start point of the single-limb support from a time of an end point of the single-limb support (t SS i=t SS end i−t SS start i).

In this case, as illustrated in FIG. 11, a time of a start point of an i-th single-limb support is the same as that of an end point of an i−1-th double-limb support (t SS start i=t DS end (i−1)), and a time of an end point of the i-th single-limb support is the same as a time of a start point of an i-th double-limb support (t SS end i=t DS start i).

Referring to FIG. 12, region A is a region in which a section of 150 to 200 ms of the gait cycle is cut, and the gait posture of the pedestrian may be analyzed using peak values located in region A.

Specifically, a first peak acceleration value 1202 indicates a point (foot-flat) at which the foot comes into contact with the ground, and is the highest acceleration value between a time 1201 (t SS start i) of a start point of the i-th single-limb support and a time 1205 (t SS mid i) of an i-th intermediate load section. In addition, a second peak acceleration value 1203 indicates a point (heel-off) at which the heel of the foot is away from the ground, and is the highest acceleration value between the time 1205 (t SS mid i) of an i-th intermediate load section and a time 1204 (t SS end i) of an end point of the i-th single-limb support. In addition, a trough acceleration value indicates a mid stance point, and is an acceleration value corresponding to t SS mid i. In this case, it is possible to find out a force value at a corresponding point-in-time by multiplying the respective values (the first peak acceleration value, the second peak acceleration value, and the trough acceleration value) by a body weight of the pedestrian.

Therefore, as illustrated in FIG. 13, the gait posture of the user may be divided using a graph illustrating a normalized ground reaction force (BW) for each gait cycle (% of gait cycle).

In addition, in FIG. 12, a cadence of the pedestrian may be calculated by taking ‘1/peak time interval’. However, the cadence may be calculated by various methods. For example, the cadence may be calculated by taking ‘1/trough time interval’, may be calculated by taking ‘1/time interval between start points of double-limb supports’, or may be calculated by taking ‘1/time interval between end points of double-limb supports’.

Generally, a system of measuring a gait motion according to the related art has collected ground reaction forces by attaching sensors to limb parts of a human body or has collected ground reaction forces separated from each other from two different force plates. Therefore, ground reaction forces for each of the left foot and the right foot are collected, and thus, a work of summing the ground reaction forces for each of the left foot and the right foot needs to be performed in order to analyze the gait posture of the pedestrian. Therefore, there was a disadvantage that work efficiency is low. Furthermore, in a case of collecting the ground reaction forces using the force plates, there was a disadvantage that portability is poor.

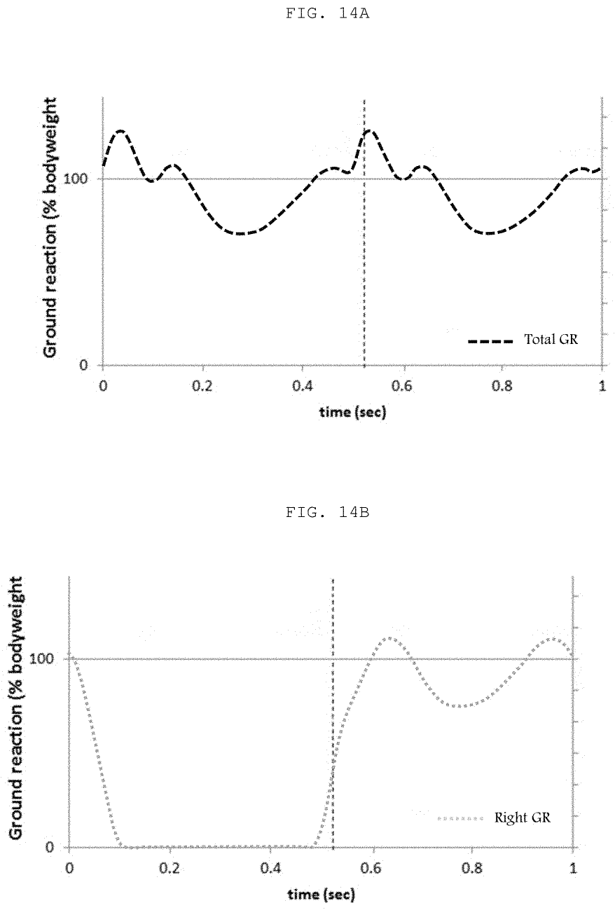

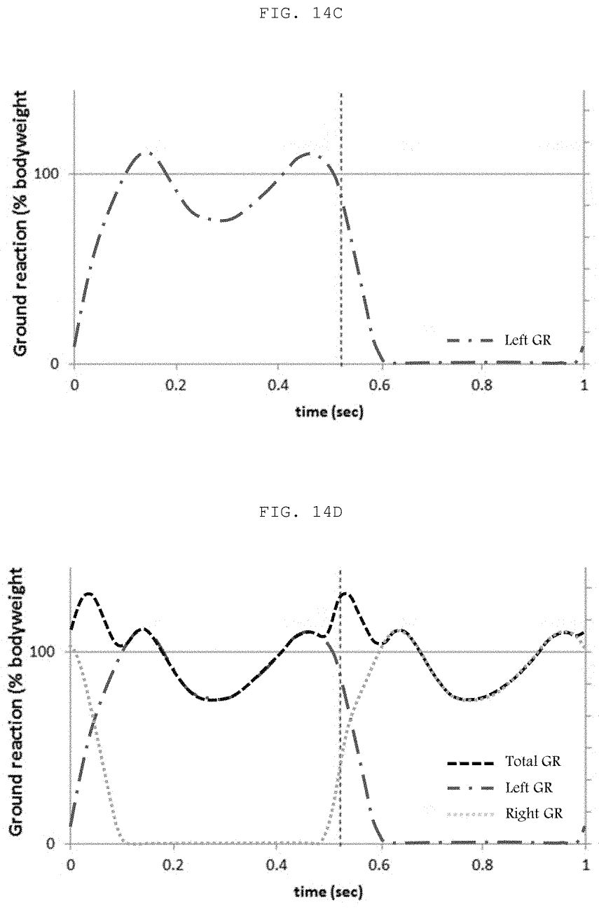

On the other hand, the present applicant has found that it is possible to analyze a gait posture of a pedestrian by attaching only an acceleration sensor to a head part of the pedestrian without using the force plates through an experiment. As illustrated in FIGS. 14A to 14D, it is possible to find the ground reaction forces of each of the right foot and the left foot using a case where the left foot and the right foot come into contact with the ground at the same time (see FIGS. 14B and 14C) after collecting a total ground reaction force (GR) of both feet through the apparatus 100 of detecting a gait parameter attached to the head part (see FIG. 14A). In addition, by using the method of detecting a gait parameter described above, it is also possible to distinguish gait motions from each other such as which section is the single-limb support and which section is the double-limb support from graphs as illustrated in FIG. 14D.

In addition, in embodiments of the present invention, only the head part acceleration sensor (that is, the apparatus 100 of detecting a gait parameter) is used to collect the gait acceleration signal 501 of the pedestrian and analyze the gait posture of the pedestrian, and it is thus possible to improve a working speed.

In addition, in embodiments of the present invention, gait points-in-time for the left foot and the right foot of the pedestrian are distinguished from each other on the basis of the gait acceleration collected from the acceleration sensor attached to the head part of the pedestrian, and it is thus possible to increase convenience of the work.

Further, in embodiments of the present invention, the vertical acceleration and/or the horizontal acceleration are collected using the apparatus 100 of detecting a gait parameter, such that it is possible to analyze a gait motion in more detail, and it is thus possible to accurately detect an abnormal gait motion of patients with Parkinson's disease or patients with Alzheimer's disease. Further, it is possible to prevent an accident of the patients with Parkinson's disease or the patients with Alzheimer's disease in advance by detecting abnormal gait motion patterns of the patients with Parkinson's disease or the patients with Alzheimer's disease. For example, it is possible to prevent a fall accident or the like by analyzing abnormal gait motion patterns of the patients.

Logical blocks, modules or units described in connection with embodiments disclosed herein can be implemented or performed by a computing device having at least one processor, at least one memory and at least one communication interface. The elements of a method, process, or algorithm described in connection with embodiments disclosed herein can be embodied directly in hardware, in a software module executed by at least one processor, or in a combination of the two. Computer-executable instructions for implementing a method, process, or algorithm described in connection with embodiments disclosed herein can be stored in a non-transitory computer readable storage medium.

Although exemplary embodiments of the present invention has been described in more detail with reference to the accompanying drawings, the present invention is not necessarily limited to these exemplary embodiments, but may be variously modified without departing from the scope and spirit of the present invention. Accordingly, exemplary embodiments disclosed in the present invention are not to limit the technical spirit of the present invention, but are to describe the technical spirit of the present invention. The scope of the technical spirit of the present invention is not limited to these exemplary embodiments. Therefore, it is to be understood that the exemplary embodiments described above are illustrative rather than being restrictive in all aspects. The scope of the present invention should be interpreted by the following claims, and it should be interpreted that all the spirits equivalent to the following claims fall within the scope of the present invention.

According to embodiments of the present invention, the head part acceleration sensor attached to the head part of the pedestrian is used to collect the gait acceleration signal of the pedestrian and analyze the gait posture of the pedestrian, and it is thus possible to improve a working speed.

In addition, according to embodiments of the present invention, the left foot and the right foot of the pedestrian are distinguished from each other within the gait cycle on the basis of the gait acceleration collected from the acceleration sensor attached to the head part of the pedestrian, and it is thus possible to increase convenience of the work.

The effects according to embodiments of the present invention are not limited by the contents exemplified in the above, and more various effects are included in the present specification.

### DETAILED DESCRIPTION OF MAIN ELEMENTS

100: apparatus of detecting gait parameter
110: gait measuring module
111: sensor module
112: sensor control module
113, 125: communication module
120: gait analyzing module
121: acceleration collecting module
122: gait point-in-time detecting module
123: gait parameter detecting module
124: analysis control module
126: user interface module
130: database
140: notifying module
501: gait acceleration signal
502: gait jerk signal
901, 903: peak of gait jerk signal

What is claimed is:

1. A method of detecting a gait parameter through a head part acceleration sensor, comprising:
a gait acceleration collecting step of collecting an acceleration in a vertical direction of a pedestrian measured by an acceleration sensor disposed at a head part of the pedestrian;
a gait point-in-time detecting step of setting a gait point-in-time by extracting a peak value in the acceleration in the vertical direction within a gait cycle including a standing phase and a swing phase and detecting a start point and an end point of a double-limb support on the basis of the peak value; and
a gait parameter detecting step of detecting the double-limb support on the basis of the start point and the end point in order to analyze a gait posture of the pedestrian,
wherein the start point and the end point of the double-limb support are set within a window predetermined on the basis of peaks of the gait cycle, the start point of the double-limb support is the same as an end point of a single-limb support, and the end point of the double-limb support is the same as a start point of a single-limb support.

2. The method of detecting a gait parameter through a head part acceleration sensor of claim 1, wherein in the gait point-in-time detecting step, a gait jerk signal at a point-in-time in which an impact is applied to a ground when a limb moves from the single-limb support to the double-limb support is further detected by differentiating the acceleration in the vertical direction, and
a peak value of the gait jerk signal located before the peak value of the acceleration in the vertical direction is set as the start point of the double-limb support, and a peak value of the gait jerk signal located after the peak value of the acceleration in the vertical direction is set as the end point of the double-limb support.

3. The method of detecting a gait parameter through a head part acceleration sensor of claim 1, wherein in the gait parameter detecting step, a cadence and a step width are detected on the basis of the peak value of the acceleration in the vertical direction, and
a gait velocity, a stride length, and a step length of the pedestrian are further detected in a case of using a global positioning system (GPS).

4. The method of detecting a gait parameter through a head part acceleration sensor of claim 3, wherein the stride length is calculated by the following Equation 1:
Stride Length=Gait Velocity/Cadence. (Equation 1)

5. The method of detecting a gait parameter through a head part acceleration sensor of claim 1, wherein the gait acceleration collecting step includes a step of collecting an acceleration in a horizontal direction of the pedestrian, and
a left foot and a right foot of the pedestrian are distinguished from each other according to signs of the acceleration in the horizontal direction.

6. The method of detecting a gait parameter through a head part acceleration sensor of claim 1, wherein in the window, after a highest peak of the peaks of the gait cycle is set as a reference peak, a window size is set to be smaller than a range between peaks disposed on both sides of the reference peak.

7. An apparatus of detecting a gait parameter through a head part acceleration sensor, comprising:
a gait measuring module measuring an acceleration in a vertical direction by a gait acceleration sensor disposed in the vicinity of a head of a pedestrian and transferring a measured value; and
a gait analyzing module including an acceleration collecting module collecting the measured value received from the gait measuring module, a gait point-in-time module extracting a peak value in the acceleration in the vertical direction on the basis of the measured value and detecting a start point and an end point of a double-limb support, and a gait parameter detecting module detecting the double-limb support on the basis of the start point and the end point,
wherein the start point and the end point of the double-limb support are set within a window predetermined on the basis of peaks of a gait cycle, the start point of the double-limb support is the same as an end point of a single-limb support, and the end point of the double-limb support is the same as a start point of a single-limb support.

8. The apparatus of detecting a gait parameter through a head part acceleration sensor of claim 7, wherein the gait point-in-time module further detects a gait jerk signal at a point-in-time in which an impact is applied to a ground when a limb moves from the single-limb support to the double-limb support by differentiating the acceleration in the vertical direction, and
a peak value of the gait jerk signal located before the peak value of the acceleration in the vertical direction is set as the start point of the double-limb support, and a peak value of the gait jerk signal located after the peak value of the acceleration in the vertical direction is set as the end point of the double-limb support.

9. The apparatus of detecting a gait parameter through a head part acceleration sensor of claim 7, further comprising a notifying module converting the gait parameter detected by the gait analyzing module into a form recognized by a user, including a sound or an image, and outputting gait posture analysis information so that the pedestrian corrects a gait posture.
